(19) United States
(12) Plant Patent Application Publication
(10) Pub. No.: US 2010/0138969 P1
(43) Pub. Date: Jun. 3, 2010

(54) Ornithogalum plant named 'Bethlehem'

(50) ;

(76) Inventor: Rodolfo Valdoz Bautista, Half Moon Bay, CA (US)

Correspondence Address: Law offices of James R. Cypher, 409 13th Street, 11th Floor, OAKLAND, CA, 94612, US

(21) Appl. No.: 12/315,215

(22) Filed: Dec. 1, 2008

(51) Int. Cl. A01H 5/00

(52) U.S. Cl. PLT/462

(57) ABSTRACT

A new and distinct cultivar of Ornithogalum thyrsoides Jacq. named ‘Bethlehem’ originated as a controlled cross between the commercial cultivar Ornithogalum thyrsoides Jacq. ‘Snowflake’ and an unnamed Ornithogalum thyrsoides Jacq. cultivar. The cultivar ‘Bethlehem’ has R.H.S. 155 C (white group) petals at maturity and is distinguished from other similar cultivars of which the inventor is aware by its tall racemes, wide leaves and suitability for growth in pots.

### BOTANICAL CLASSIFICATION

Ornithogalum thyrsoides Jasq. ‘Bethlehem’

### VARIETY DENOMINATION

‘Bethlehem’

### BACKGROUND OF THE INVENTION

This invention relates to a new and distinct cultivar of the Lilaceae family. The botanical name of the plant is Ornithogalum thyrsoides Jasq. ‘Bethlehem’.

The new cultivar originated as a seedling from a controlled cross between the commercial variety Ornithogalum thyrsoides ‘Snowflake’ and an unnamed Ornithogalum thyrsoides. ‘Snowflake’ was the seed parent. ‘Snowflake’ is the progeny of the commercial variety ‘Bock Bay’.

The variety ‘Bethlehem’ is most like its parent ‘Snowflake’.

The new variety was first noticed, because of its erect and tall racemes and its large flowers.

The new cultivar ‘Bethlehem’ has been asexually reproduced through three generations under controlled environmental conditions at a nursery in Half Moon Bay, Calif. under the direction of the inventor with its distinguishing characteristics remaining stable.

‘Bethlehem’ is distinguished from other Ornithogalum thyrsoides varieties of which the inventor is aware by its tall, upright racemes, its wide leaves, its suitability for growth in pots and its large flowers.

Asexual reproduction was first accomplished when vegetative cuttings were taken from the initially selected plant. Examination of asexually reproduced, successive generations grown in a nursery at Half Moon Bay, Calif. show that the combination of characteristics as herein disclosed for ‘Bethlehem’ remains firmly fixed through three generations.

### DESCRIPTION OF THE DRAWINGS

The accompanying drawings consist of color photographs that show the typical plant form, including the inflorescence, foliage, and sepals.

FIG. 1 is a view of the new variety showing its form, upright growth habit, dark green foliage, inflorescence, and non-pigmented petals.

FIG. 2 is a view of the inflorescence of the new variety.

FIG. 3 is a close-up view of the inflorescence of the new variety, showing opened florets, and opening florets.

FIG. 4 is a close-up view of the inflorescence of the new variety, showing opened florets, and opening florets.

FIG. 5 is a top view of a number of inflorescences of the new variety.

FIG. 6 is a close-up view of the base of the raceme of the new variety.

FIG. 7 is a view of the base of the new variety.

FIG. 8 is a close up view of the upper portion of an inflorescence of the new variety.

FIG. 9 is a top view of the new variety.

FIG. 10 is a top view of an inflorescence of the new variety.

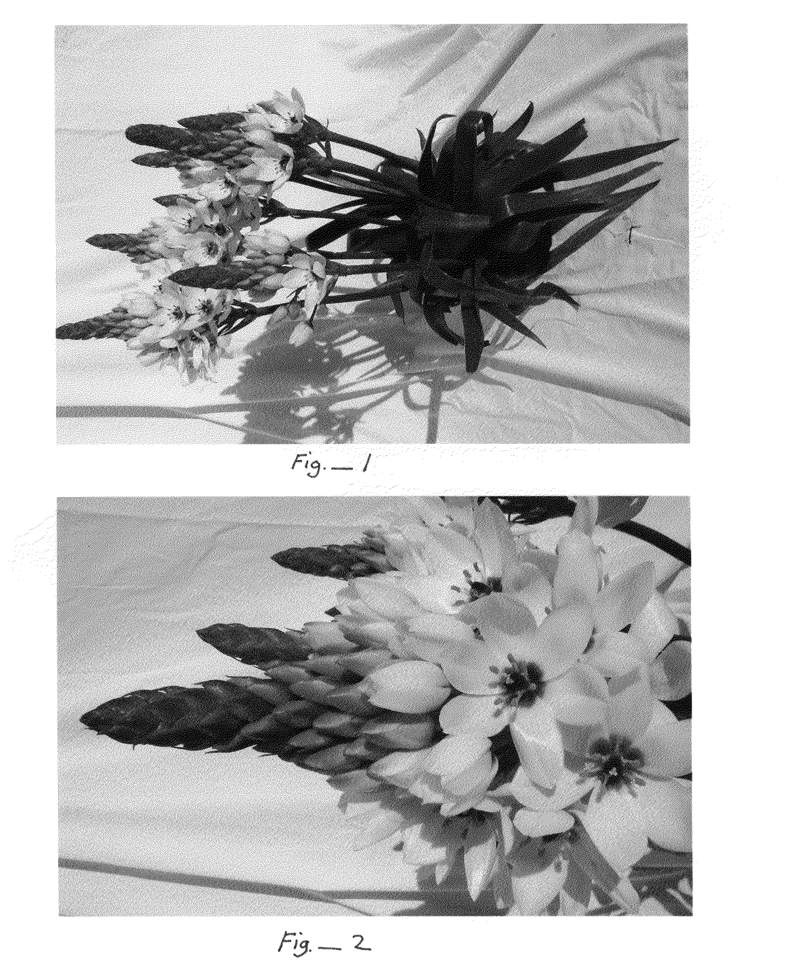

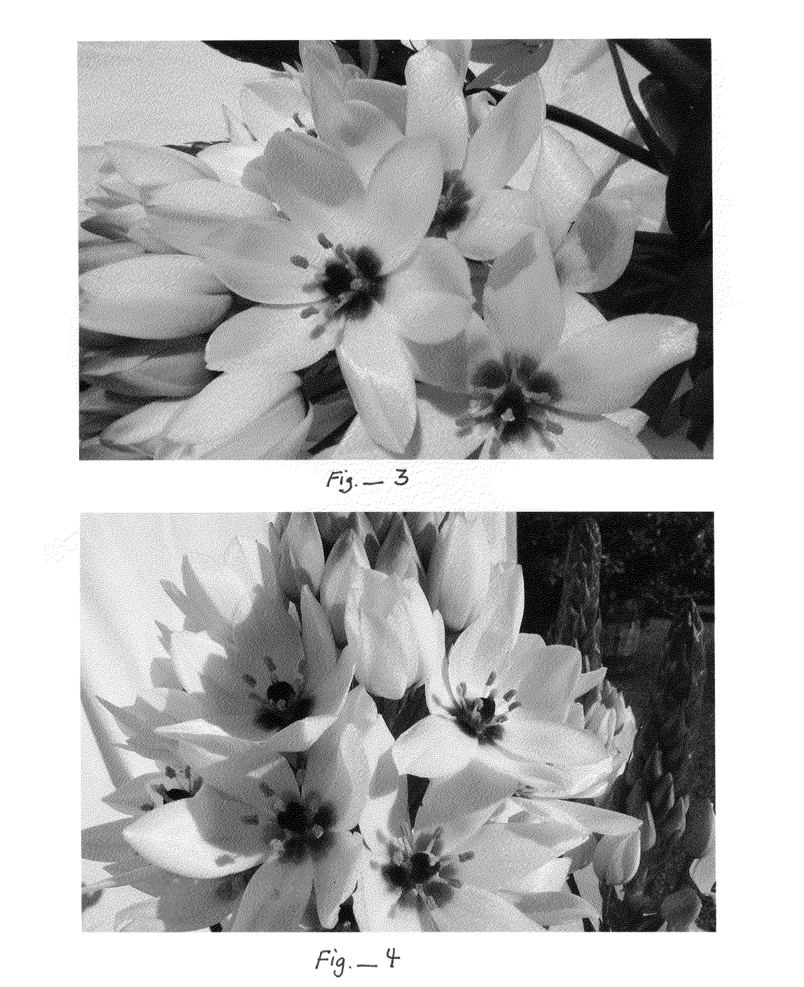

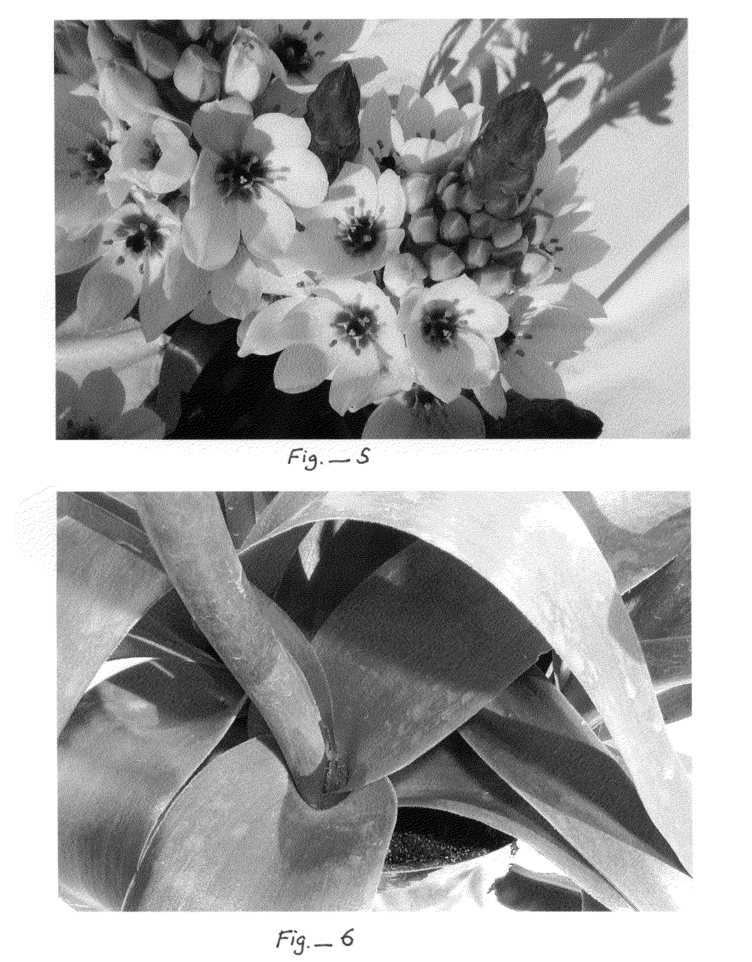

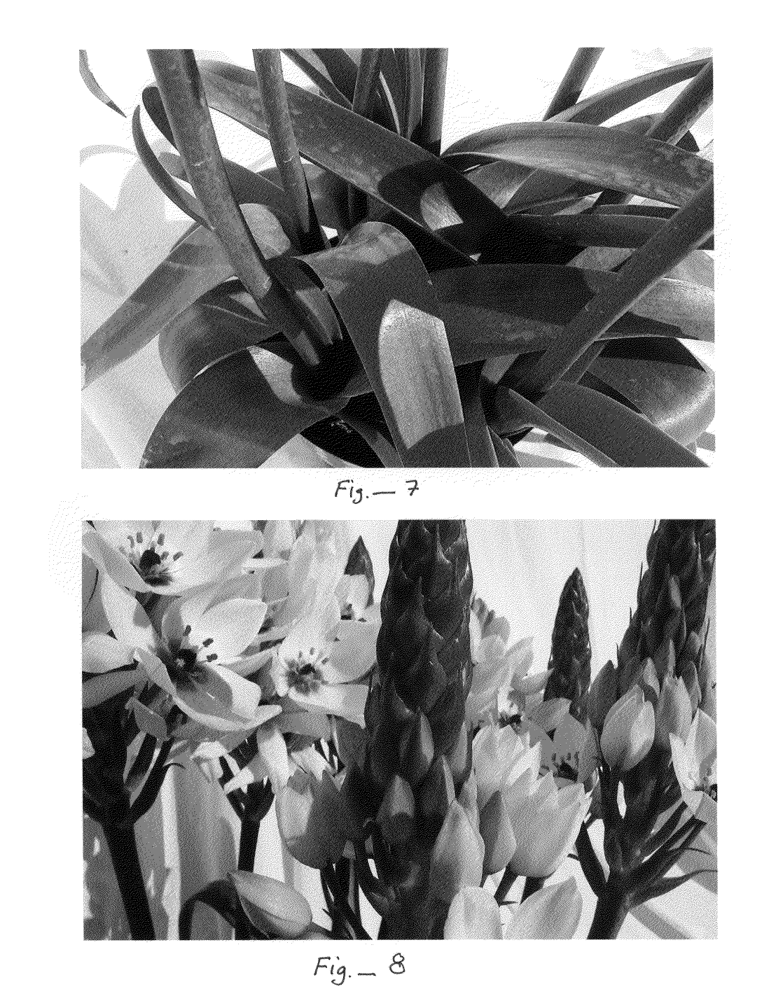

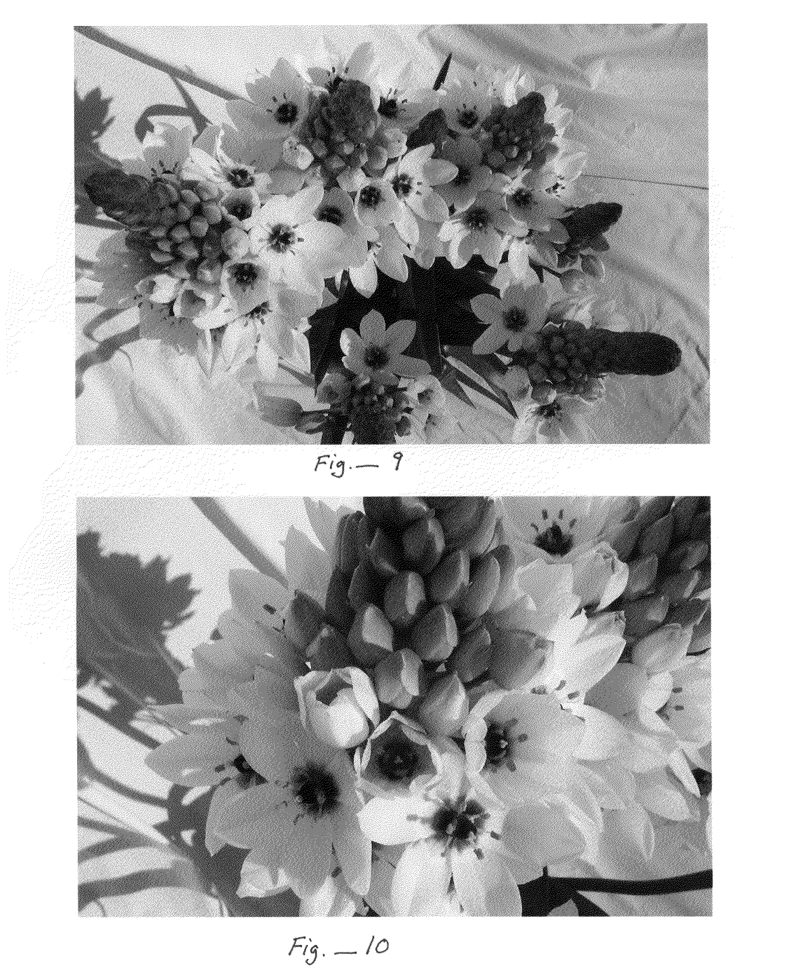

### DESCRIPTION OF THE NEW PLANT

The plants shown in the figures were grown in 6″ pots.

‘Bethlehem’ has not been observed under all possible environmental conditions. The phenotype may vary significantly with variations in environment such as temperature, light intensity and day length. Color determinations were made with The Royal Horticultural Society (R.H.S.) Colour Chart.
The plant:
   Origin.—Controlled cross.
   Parents.—Ornithogalum thyrsoides Jasq. ‘Snowflake’ and an unnanmed Ornithogalum thyrsoides Jasq.
   Botanic classification.—Ornithogalum thyrsoides Jasq. ‘Bethlehem’.
   Form.—Spreading basal leaves with upright inflorescences on tall rachis. A typical plant with a mature inflorescence that is ready for sale is approximately 43 cm high when grown in a 6″ pot with appropriate soil amendments.
   Growth.—Upright rachis with spreading leaves. Inflorescence is large and showy.
   Rachis.—The rachis is upright and glaucescent. Its color is R.H.S. 146 A (yellow-green group).
   Foliage.—Usually five leaves on a mature plant ready for commercial sale. Leaves are whorled at base of stem.
   Shape of leaf.—Fleshy, varying from channeled to flat, lanceolate. Margins are entire.
   Texture.—Glaucascent.
   Color.—Mature leaves are R.H.S. 146 A (yellow-green group). Large leaves are 25 cm long and 4 cm wide.
Inflorescence:
   Form.—Corymbose racemes. Indeterminate As many as 100 or more individual flowers (florets) per inflorescence on pedicels that elongate as the florets opens, and can be as long as 56 mm. Flowers do not produce a fragrance. The rachis for the inflorescence is strong and upright. The pedicels are predominately green — R.H.S. 146 A (yellow-green group) at maturity.
   Size of inflorescence.—Large. A typical inflorescence shown in the figures can be as large as 15 cm in diameter, and 23 cm high.
   Shape.—Basal florets opening first with pedicels that elongate that give the raceme a corymbose appearance as the inflorescence ages.
   Appearance.—Showy.
Florets:
   General.—The florets at the base of the inflorescence open first, and open more and more as the inflorescence ages. Florets are perfect and complete. Perianth is not keeled. Each floret is associated with a bract that can be as high as 50 mm and is same color as leaves — R.H.S. 146 A (yellow-green group).
   Corolla.—There are 6 petals that are spotted at their base — R.H.S. 152 A (yellow-green group), but predominately white — R.H.S. 155 C (white group) with the mid-vein showing on the abaxial surface.
   Flower diameter.—70 mm. Petals are typically 36 mm high, 17 mm wide, 4 mm wide at their attachment, and the spotted base is approximately 5 mm high and extends the width of the petal at the base.
   Stamens.—6 stamens (two sets of 3). Pollen is R.H.S. 17 A (yellow-green group). Plant produces abundant pollen. Filament is flattened with translucent wings. Filament is 3 mm wide at base and 10 mm high. Filament is 3 mm at its widest point. Anther is 4 mm high and basally attached — R.H.S. 69 A (red-purple group) and approximately 4 mm long. Anther is 1 mm long and is regular and basally attached.
   Stigma.—Generally, a three-pronged stigma on one pistil per floret. The style is 2 mm long. The stigma is approximately 2 mm long.
   Ovary.—The ovary is three-celled with axile placentae. The base of the ovary is R.H.S. 152 C (yellow-green group) and the top of the ovary is R.H.S. 200 B (brown group). The ovary is 6 mm high and has a diameter of 7 mm.

I claim:

1. A new and distinct Ornithogalum plant named ‘Bethlehem’ substantially as herein shown and described.
